(19) United States
(12) Patent Application Publication
(10) Pub. No.: US 2004/0006077 A1
(43) Pub. Date: Jan. 8, 2004

(54) Thiazine and oxazine derivatives as MMP-13 inhibitors

(76) Inventors: Bernard Gaudilliere, Nanterre (FR); Henri Jacobelli, Paray Vieille Poste (FR)

Correspondence Address: WARNER-LAMBERT COMPANY, 2800 PLYMOUTH RD, ANN ARBOR, MI, 48105, US

(21) Appl. No.: 10/602,160

(22) Filed: Jun. 24, 2003

Related U.S. Application Data
(60) Provisional application No. 60/391,374, filed on Jun. 25, 2002.

(30) Foreign Application Priority Data
Jun. 25, 2002 (WO) PCT/EP02/08062

(51) Int. Cl. A61K 31/541; A61K 31/5377

(52) U.S. Cl. 514/227.8; 514/231.5

(57) ABSTRACT

The specification discloses compounds selected from those of formula (I):

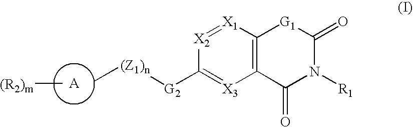

or a racemic form, isomer, N-oxide, or pharmaceutically acceptable salt thereof, wherein R₂, m, A, Z₁, n, G₂, X₁, X₂, X₃, G₁, and R₁ are as defined in the specification, wherein the compound of formula (I) is not 6-(2,4-dioxo-3,4-dihydro-2H-1,3-benzothiazine)-benzoate, 6-phenylthio-2,4-dioxo-3,4-dihydro-2H-1,3-benzothiazine, 6-benzylsulphonyl-2,4-dioxo-3,4-dihydro-2H-1,3-benzothiazine, 6-benzophenone-2,4-dioxo-3,4-dihydro-2H-1,3-benzothiazine or 6-(2,4-dihydroxy)-benzophenone-2,4-dioxo-3,4-dihydro-2H-1,3-benzothiazine. The specification also discloses processes for preparing the compounds, pharmaceutical compositions comprising said compounds of formula (I) or a racemic form, isomer, N-oxide, or pharmaceutically acceptable salt thereof, and methods of treating diseases or disorders mediated by a MMP-13 enzyme or responsive to treatment with an inhibitor of a MMP-13 enzyme, comprising administering said compounds of formula (I) or a racemic form, isomer, N-oxide, or pharmaceutically acceptable salt thereof.

### CROSS-REFERENCE TO RELATED APPLICATIONS

This application claims benefit of priority from United States Provisional Patent Application No. 60/391,374, and PCT International Patent Application Number PCT/EP02/08062, each filed on Jun. 25, 2002.

The present invention relates to novel thiazine and oxazine derivatives which are useful for treating diseases and disorders mediated by a matrix metalloprotease-13 (MMP-13) enzyme, processes for preparing the derivatives, and methods for treating certain arthritic conditions such as rheumatoid arthritis or osteoarthritis, as well as certain proliferative conditions such as cancers with the derivatives.

### BACKGROUND

Matrix metalloproteases (MMPs) are enzymes which are involved in the renewal of extracellular matrix tissue, such as cartilage, tendons and joints. MMPs bring about the destruction of the extracellular matrix tissue, which is compensated for, in a non-pathological physiological state, by its simultaneous regeneration.

Under normal physiological conditions, the activity of these extremely aggressive peptidases is controlled by specialized proteins which inhibit MMPs, such as the tissue inhibitors of metalloprotease (TIMPs).

Local equilibrium of the activities of MMPs and of TIMPs is critical for the renewal of the extracellular matrix. Modifications of this equilibrium which result in an excess of active MMPs, relative to their inhibitor, induce a pathological destruction of cartilage, which is observed in particular in rheumatoid arthritis and in osteoarthritis.

In pathological situations, a degradation of articular cartilage takes place, as is the case in rheumatic diseases such as rheumatoid arthritis or osteoarthritis. In these pathologies, the cartilage degradation process predominates, leading to a destruction of the tissue and resulting in a loss of function.

At least twenty different matrix metalloproteases have been identified to date and are subdivided into four groups, the collagenases, the gelatinases, the stromelysins and the membrane-type MMPs (MT-MMPs), respectively.

Matrix metalloprotease-13 (MMP-13) is a collagenase-type MMP which has been shown to mediate pathologies of rheumatoid arthritis, osteoarthritis, in the course of which pathology the chondrocyte directs the destruction of cartilage, and breast cancer.

There is a need in the prior art for novel MMP inhibitors, more particularly for MMP-13 inhibitors, in order to prevent and/or correct the imbalance in the renewal of extracellular matrix tissue, such as arthritis, rheumatoid arthritis, osteoarthritis, osteoporosis, periodontal diseases, inflammatory bowel disease, psoriasis, multiple sclerosis, cardiac insufficiency, atherosclerosis, asthma, chronic obstructive pulmonary diseases (COPD), age-related macular degeneration (ARMD) and cancer.

MMP-inhibitor compounds are known. Most of these MMP-inhibitors are not selective for a single MMP, such as those described by Montana and Baxter (2000) or by Clark et al. (2000).

There is also a need in the prior art for novel inhibitors that are active on matrix metalloprotease-13, in order to enrich the therapeutic arsenal that can be used for treating pathologies associated with the destruction of the extracellular matrix and with cancer.

### SUMMARY OF THE INVENTION

The applicant has identified novel thiazine and oxazine derivatives that are matrix metalloprotease inhibitors, and more specifically compounds that are selective MMP-13 inhibitors.

In one aspect, the present invention relates to compounds of formula (I):

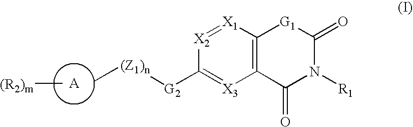

wherein:

X₁, X₂, and X₃, independently of each other, represent a nitrogen atom or a group —CR₃ in which R₃ represents a group selected from hydrogen, (C₁-C₆)alkyl, amino, mono(C₁-C₆)alkylamino, di(C₁-C₆)alkyl amino, hydroxy, (C₁-C₆)alkoxy, and halogen, it being understood that not more than two of the groups X₁, X₂ and X₃ simultaneously represent a nitrogen atom,

G₁ represents an oxygen atom or a group S(O)ₚ in which p represents an integer from 0 to 2 inclusive,

G₂ represents a group selected from carbon-carbon triple bond, C═O, C═S, S(O)_{q} in which q represents an integer from 0 to 2 inclusive, or a group of formula (i/a):

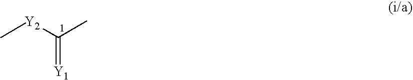

in which the carbon atom with the number 1 is attached to the bicycle of the compound of formula (I), Y₁ represents a group selected from oxygen, sulphur, —NH and —N(C₁-C₆)alkyl, and Y₂ represents a group selected from oxygen, sulphur, —NH and —N(C₁-C₆)alkyl,

n represents an integer from 0 to 6 inclusive,

Z₁ represents —CR₄R₅, wherein R₄ and R₅, identical or different independently of each other, represent a group selected from hydrogen, (C₁-C₆)alkyl, trihalogeno(C₁-C₆)alkyl, halogen, amino, mono(C₁-C₆)alkylamino, di(C₁-C₆)alkylamino in which each alkyl moiety is identical or different, —OR₆, —SR₆, and —C(═O)OR₆, in which R₆ is hydrogen atom or (C₁-C₆)alkyt, and

wherein when n is greater than or equal to 2, the hydrocarbon chain Z₁ optionally contains one or two isolated or conjugated multiple bonds,

and/or wherein when n is greater than or equal to 2 one of said —CR₄R₅ may be replaced with a group selected from oxygen, S(O)ᵣ in which r represents an integer from 0 to 2 inclusive, —NH and —N(C₁-C₆)alkyl,

A represents a group selected from aryl, heteroaryl, cycloalkyl, and heterocycloalkyl, these groups being a 5- or 6-membered monocycle, or bicycle itself composed of two 5- or 6-membered monocycles,

R₁ represents a group selected from:

hydrogen,

(C₁-C₆)alkyl, (C₂-C₆)alkenyl, (C₂-C₆)alkynyl, these groups may be optionally substituted with one or more groups, which may be identical or different independently of each other, selected from amino, cyano, trihalogeno(C₁-C₆)alkyl, cycloalkyl, —C(═O)NR₇R₈, —C(═O)OR₇, OR₇, and SR₇, in which R₇ and R₈, which may be identical or different independently of each other, represent hydrogen or (C₁-C₆)alkyl,

and the group of formula (i/b):

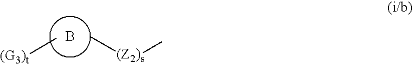

in which s is an integer from 0 to 8 inclusive,

Z₂ represents —CR₉R₁₀ wherein R₉ and R₁₀, identical or different independently of each other, represent a group selected from hydrogen, (C₁-C₆)alkyl, phenyl, trihalogeno(C₁-C₆)alkyl, halogen, amino, OR₆, SR₆ and —C(═O)OR₆ in which R₆ is as defined hereinbefore, and

wherein when s is greater than or equal to 2, the hydrocarbon chain Z₂ optionally contains one or two isolated or conjugated multiple bonds,

and/or wherein when p is greater or equal to 2, one of said —CR₉R₁₀ may be replaced with a group selected from oxygen, S(O)ᵤ in which u is an integer from 0 to 2 inclusive, —NH, —N(C₁-C₆)alkyl, and carbonyl,

B represents a group selected from aryl, heteroaryl, cycloalkyl, and heterocycloalkyl, these groups being a 5- or 6-membered monocycle, or bicycle itself composed of two 5- or 6-membered monocycles,

t is an integer from 0 to 7 inclusive,

the group(s) G₃, which may be identical or different independently of each other, is (are) selected from (C₁-C₆)alkyl, halogen, CN, NO₂, CF₃, OCF₃, —(CH₂)ₖNR₁₁R₁₂, —N(R₁₁)C(═O)R₁₂, —N(R₁₁)C(═O)OR₁₂, —N(R₁₁)SO₂R₁₂, —N(SO₂R₁₁)₂, —OR₁₁, —S(O)ₖ₁R₁₁, —SO₂—N(R₁₁)—(CH₂)ₖ₂—NR₁₂R₁₃, —(CH₂)ₖSO₂NR₁₁R₁₂, —X₄(CH₂)ₖC(═O)OR₁₁, —(CH₂)ₖC(O)OR₁₁, —C(═O)O—(CH₂)ₖ₂—NR₁₁R₁₂, —C(═O)O—(CH₂)ₖ₂—C(═O)OR₁₄, —X₄(CH₂)ₖC(═O)NR₁₁R₁₂, —(CH₂)ₖC(═O)NR₁₁R₁₂, —R₁₅—C(═O)OR₁₁, —X₅—R₁₆, and —C(═O)—R₁₇—NR₁₁R₁₂ in which:

X₄ represents a group selected from oxygen, sulphur optionally substituted by one or two oxygen, and nitrogen substituted by a hydrogen or a (C₁-C₆)alkyl group,

k is an integer from 0 to 3 inclusive,

k1 is an integer from 0 to 2 inclusive,

k2 is an integer from 1 to 4 inclusive,

R₁₁, R₁₂ and R₁₃, which may be identical or different independently of each other, are selected from hydrogen and (C₁-C₆)alkyl,

R₁₄ represents a group selected from (C₁-C₆)alkyl, —R₁₇—NR₁₁R₁₂, —R₁₇—NR₁₁—C(═O)—R₁₇—NR₁₂R₁₃, and —C(═O)O—R₁₇—NR₁₁R₁₂ in which R₁₇ represents a linear or branched (C₁-C₆)alkylene group, and R₁₁, R₁₂ and R₁₃ are as defined hereinbefore,

R₁₅ represents a (C₃-C₆)cycloalkyl group,

X₅ represents a group selected from a single bond, —CH₂—, oxygen, sulphur optionally substituted by one or two oxygen, and nitrogen substituted by hydrogen or (C₁-C₆)alkyl,

R₁₆ represents a group selected from:

a 5- or 6-membered monocyclic aryl or heteroaryl, which is optionally substituted by one or more groups, which may be identical or different independently of each other, selected from (C₁-C₆)alkyl, halogen, hydroxy, cyano, tetrazolyl, amino, and —C(═O)OR₇ wherein R₇ represents hydrogen or (C₁-C₆)alkyl,

and a 5- or 6-membered monocyclic cycloalkyl or heterocycloalkyl, which is optionally substituted by one or more groups, which may be identical or different independently of each other, selected from (C₁-C₆)alkyl, halogen, hydroxy, oxo, cyano, tetrazolyl, amino, and —C(═O)OR₇ wherein R₇ represents hydrogen or (C₁-C₆)alkyl,

m is an integer from 0 to 7 inclusive,

the group(s) R₂, which may be identical or different independently of each other, is (are) selected from (C₁-C₆)alkyl, halogen, —CN, —NO₂, —SCF₃, —CF₃, —OCF₃, —NR₇R₈, —OR₇, —SR₇, —SOR₇, —SO₂R₇, —(CH₂)ₖSO₂NR₇R₈, —X₇(CH₂)ₖC(═O)OR₇, —(CH₂)ₖC(═O)OR₇, —X₇(CH₂)ₖC(═O)NR₇R₈, —(CH₂)ₖC(═O)NR₇R₈, and —X₈—R₁₈ in which:

X₇ represents a group selected from oxygen, sulphur optionally substituted by one or two oxygen, and nitrogen substituted by hydrogen or (C₁-C₆)alkyl,

k is an integer from 0 to 3 inclusive,

R₇ and R₈, which may be identical or different independently of each other, are selected from hydrogen and (C₁-C₆)alkyl,

X₈ represents a group selected from single bond, —CH₂—, oxygen, sulphur optionally substituted by one or two oxygen, and nitrogen substituted by hydrogen or (C₁-C₆)alkyl,

R₁₈ represents a group selected from phenyl, a 5- or 6-membered monocyclic, heteroaryl, and a 5- or 6-membered monocyclic cycloalkyl, each of these groups being optionally substituted by one or more groups, which may be identical or different independently of each other, selected from (C₁-C₆)alkyl, halogen, hydroxy and amino,

and optionally, their racemic forms, isomers, N-oxides, and pharmaceutically acceptable salts,

wherein the compound of formula (I) is not 6-(2,4-dioxo3,4-dihydro-2H-1,3-benzothiazine)-benzoate, 6-phenylthio-2,4-dioxo-3,4-dihydro-2H-1,3-benzothiazine, 6-benzylsulphonyl-2,4-dioxo-3,4-dihydro-2H-1,3-benzothiazine, 6-benzophenone-2,4-dioxo-3,4-dihydro-2H-1,3-benzothiazine or 6-(2,4-dihydroxy)-benzophenone-2,4-dioxo-3,4-dihydro-2H-1,3-benzothiazine.

According to another aspect, the invention relates to compounds of formula (I) wherein:

G₁ represents a sulphur atom,

G₂ represents a group of formula (i/a):

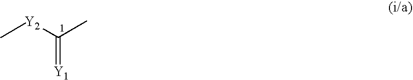

in which the carbon atom with the number 1 is attached to the bicycle of the compound of formula (I), Y₁ represents an oxygen atom, and Y₂ represents a group —NH,

X₁, X₂, X₃, n, Z₁, A, R₁, m and R₂ are as defined in formula (I).

According to another aspect, the invention relates to compounds of formula (I) wherein:

G₁ represents an oxygen atom,

G₂ represents a group of formula (i/a):

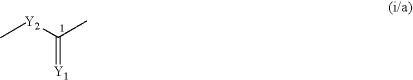

in which the carbon atom with the number 1 is attached to the bicycle of the compound of formula (I), Y₁ represents an oxygen atom, and Y₂ represents a group —NH,

X₁, X₂, X₃, n, Z₁, A, R₁, m and R₂ are as defined in formula (I).

According to another aspect, the invention relates to compounds of formula (I) wherein

G₁ represents a sulphur atom,

G₂ represents a carbon-carbon triple bond,

n represents an integer from 1 to 6 inclusive,

X₁, X₂, X₃, Z₁, A, R₁, m and R₂ are as defined in formula (I).

According to another aspect, the invention relates to compounds of formula (I) wherein:

G₁ represents an oxygen atom,

G₂ represents a carbon-carbon triple bond,

n represents an integer from 1 to 6 inclusive,

X₁, X₂, X₃, Z₁, A, R₁, m and R₂ are as defined in formula (I).

The substituent R₁ in another aspect of the invention is the group of formula (i/b):

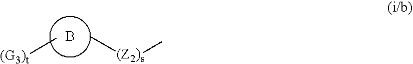

wherein Z₂, s, B, G₃ and t are as defined in the compound of formula (I).

More particularly, the substituent R₁ in another aspect of the invention is the group of formula (i/b):

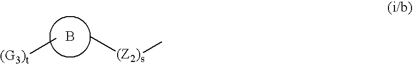

wherein Z₂ represents a group —CR₉R₁₀ in which R₉ and R₁₀ represents each a hydrogen atom, s is equal to one, and B, G₃, and t are as defined in the compound of formula (I).

More particularly, the substituent R₁ in another aspect of the invention is the group of formula (i/b):

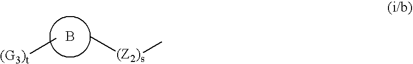

wherein B represents a phenyl group, t is equal to 0 or 1, and G₃, when it is present, represents a group selected from OR₁₁, halogen, and (CH₂)ₖC(═O)OR₁₁ in which R₁₁ represents an hydrogen atom or a (C₁-C₆)alkyl group and k is equal to zero.

In another aspect, compounds of the invention are compounds of formula (I) wherein X₁, X₂, and X₃ represent each a group —CR₃ in which R₃ represents a hydrogen atom.

In another aspect, compounds of the invention are compounds of formula (I) wherein X₁ represents a group —CR₃ in which R₃ represents a hydrogen atom, X₂ represents a nitrogen atom, and X₃ represents a group —CR₃ in which R₃ represents a hydrogen atom.

In another aspect, compounds of the invention are those compounds of formula (I) wherein Z₁ represents —CR₄R₅ in which R₄ and R₅ represent each a hydrogen atom, and n is equal to one.

In another aspect, compounds of the invention are compounds of formula (I) wherein A represents a phenyl group , m is equal to zero or one, and R₂ represents a (C₁-C₆)alkoxy group or a hydrogen atom.

In another aspect, compounds of the invention are compounds of formula (I) wherein A represents a pyridyl group, m is equal to zero or one, and R₂ represents a (C₁-C₆)alkoxy group or a hydrogen atom.

In another aspect, compounds of the invention are compound of formula (I) wherein A represents an imidazolyl group.

In another aspect, the invention is a compound of formula (I) selected from:

3-benzyl-2,4-dioxo-3,4-dihydro-2H-benzo[e][1,3]thiazine-6-carboxylic acid 4-methoxy benzylamide;

3-(4-methoxybenzyl)2,4-dioxo-3,4-dihydro-2H-benzo[e][1,3]oxazine-6-carboxylic acid 4-methoxybenzylamide; and

and 4-[2,4-dioxo-6-(3-phenyl-prop-1-ynyl)-4H-1,3-benzothiazin-3-ylmethyl]-benzoic acid; or

a pharmaceutically acceptable salt thereof.

The optical isomers, the N-oxides, as well as the pharmaceutically acceptable addition salts with an acid or base, form an integral part of the invention.

The invention also relates to a pharmaceutical composition comprising as active ingredient an effective amount of a compound of formula (I) together with one or more pharmaceutically acceptable excipients or carriers.

Another aspect of the invention concerns the use of the compound of formula (I) for the preparation of (a medicinal product intended for treating a disease involving therapy by inhibition of matrix metalloprotease, and more particularly of type-13 matrix metalloprotease.

The invention also relates to a method for treating a patient afflicted with a disease or disorder that is pathologically mediated by a type-13 matrix metalloprotease, said method comprising the administration of an effective amount of a compound of formula (I) to the patient in need thereof.

One aspect of the method of treatment according to this invention is treatment of a disease or disorder selected from arthritis, rheumatoid arthritis, osteoarthritis, osteoporosis, periodontal diseases, inflammatory bowel disease, psoriasis, multiple sclerosis, cardiac insufficiency, atherosclerosis, asthma, chronic obstructive pulmonary diseases, age-related degeneration and cancers.

Another aspect of this invention is the method of treatment according to this invention wherein the disease is arthritis. In still another aspect of this invention method, the disease is osteoarthritis or rheumatoid arthritis.

Another aspect of this invention is one of the methods of treating according to this invention wherein the compound of formula (I) being administered is 6-(2,4-dioxo-3,4-dihydro-2H-1,3-benzothiazine)-benzoate, 6-phenylthio-2,4-dioxo-3,4-dihydro-2H-1,3-benzothiazine, 6-benzylsulphonyl-2,4-dioxo-3,4-dihydro-2H- 1,3-benzothiazine, 6-benzophenone-2,4-dioxo-3,4-dihydro-2H-1,3-benzothiazine or 6-(2,4-dihydroxy)-benzophenone-2,4-dioxo-3,4-dihydro-2H-1,3-benzothiazine.

### DETAILED DESCRIPTION OF THE INVENTION

The compounds provided by this invention are those defined in formula (I). In formula (I), it is understood that:

a (C₁-C₆)alkyl group denotes a linear or branched group containing from 1 to 6 carbon atoms; example of such groups, without implying any limitation are methyl, ethyl, propyl, isopropyl, tert-butyl, neopentyl, hexyl,

a (C₂-C₆)alkenyl group denotes a linear or branched group containing from 2 to 6 carbon atoms, and one or more double bonds; examples of such groups without implying any limitation are vinyl, allyl, 3-buten-1-yl, 2-methyl-buten-1-yl, hexenyl,

a (C₂-C₆)alkynyl group denotes a linear or branched group containing from 2 to 6 carbon atoms, and one or more triple bonds; examples of such groups without implying any limitation are ethynyl, propynyl, 3-butyn-1-yl, 2-methyl-butyn-1-yl, hexynyl,

a (C₁-C₆)alkoxy group means the alkyl group as mentioned above bound through an oxygen atom; examples of such compounds without implying any limitation are methoxy, ethoxy, n-propyloxy, tert-butyloxy,

a mono(C₁-C₆)alkylamino denotes a amino group substituted by one (C₁-C₆)alkyl group as defined hereinbefore; example of such groups, without implying any limitation are methyl amino, isobutyl amino, ethylamino,

a di(C₁-C₆)alkylamino denotes a amino group substituted by two (C₁-C₆)alkyl groups as defined hereinbefore, each alkyl group being identical or different; example of such groups, without implying any limitation are dimethylamino, diethylamino,

an aryl group denotes an aromatic monocyclic or bicyclic system containing from 6 to 10 carbon atoms, and in the case of a bicyclic system, one of the ring of which is aromatic in character, and the other ring of which may be aromatic or partially hydrogenated; examples of such groups without implying any limitation are, phenyl, naphthyl, indenyl, benzocyclobutenyl,

a heteroaryl group denotes an aryl group as described above in which 1 to 4 carbon atoms are replaced by 1 to 4 hetero atoms selected from oxygen, sulfur and nitrogen, wherein two O or two S or one O and one S atoms are not contiguous; examples of such groups without implying any limitation are furyl, thienyl, pyrrolyl, pyrazolyl, pyridyl, pyrimidyl, pyrazinyl, benzofuryl, benzothienyl, indolyl, quinolyl, isoquinolyl, imidazolyl, benzodioxolyl, benzodioxinyl, benzo[1,2,5]thiadiazolyl, benzo[1,2,5]oxadiazolyl,

a cycloalkyl group denotes a monocyclic or bicyclic system containing from 3 to 10 carbon atoms, this system being saturated or partially unsaturated but without aromatic character; examples of such groups without implying any limitation are cyclopropyl, cyclobutyl, cyclopentyl, cyclohexyl, cyclooctyl, cycloheptyl, adamantyl, decalinyl, norbornyl,

a heterocycloalkyl group denotes a cycloalkyl group as defined hereinbefore in which 1 to 4 carbon atoms are replaced by 1 to 4 hetero atoms selected from oxygen, sulfur, and nitrogen, wherein two O or two S or one O and one S atoms are not contiguous;

a bicycle denotes two fused-monocycle or two bridged-monocycle,

a trihalogeno(C₁-C₆)alkyl group denotes an alkyl group as defined above which contains a trihalogeno group; examples of such groups without implying any limitation are trifluoromethyl, 2,2,2-trifluoroethyl,

a (C₁-C₇)acyl group denotes an alkyl group or an aryl group as defined above bound through a carbonyl group; examples of such groups without implying any limitation are acetyl, ethylcarbonyl, benzoyl,

a multiple bond denotes double bond or triple bond,

a halogen atom means fluoro, chloro, bromo or iodo,

optical isomers refer to racemates, enantiomers and diastereoisomers.

The term “isomer” includes stereoisomers, geometric isomers (e.g., syn, anti, cis, trans, entgegen, zusammen), tautomers, and the like.

The term “patient” means a mammal. The patient being treated will have a disease or disorder that is pathologically mediated by a MMP-13 enzyme or is responsive to treatment with an inhibitor of a MMP-13 enzyme. The mammals include a human, dog, cat, cow, pig, horse, sheep, goat, rat, mouse, rabbit, guinea pig, monkey, chimpanzee, and the like.

An “effective amount” as the phrase relates to treatment of a disease or disorder described herein means an amount that is sufficient to therapeutically alleviate, in whole or in part, at least one symptom or inhibit, in whole or in part, a pathological progression of the disease or disorder being treated.

The invention also relates to the pharmaceutically acceptable salts of the compounds of formula (I). A review of the pharmaceutically acceptable salts will be found in J. Pharm. Sci., 1977, 66, 1-19.

Pharmaceutically acceptable acids mean non-toxic mineral or organic acids. Among those there may be mentioned, without implying any limitation, hydrochloric acid, hydrobromic acid, sulfuric acid, phosphonic acid, nitric acid, citric acid, acetic acid, trifluoroacetic acid, lactic acid, pyravic acid, malonic acid, succinic acid, glutaric acid, fumaric acid, tartaric acid, maleic acid, ascorbic acid, oxalic acid, methanesulfonic acid, camphoric acid, benzoic acid, toluenesulfonic acid, etc . . .

Pharmaceutically acceptable bases mean non-toxic mineral or organic bases. Among those, there may be mentioned, without implying any limitation, sodium hydroxide, potassium hydroxide, calcium hydroxide, triethylamine, tert-butylamine, dibenzylethylenediamine, piperidine, pyrrolidine, benzylamine, quaternary ammonium hydroxides etc . . .

The invention also relates to a process for the preparation of compounds of formula (I), which uses as starting material a compound of formula (II):

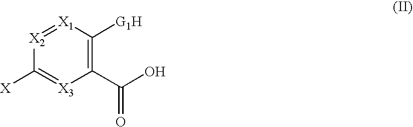

in which X₁, X₂, X₃, and G₁ have the same definitions as the compound of formula (I), and X represents a leaving group selected from halogen, triflate, mesyslate, tosylate and SO₂alkyl,

compound of formula (II) which is treated in basic medium with an isocyanate compound of formula (III):

R₁—NCO (III)

in which R₁ has the same definitions as the compound of formula (I),

to yield the compound of formula (IV):

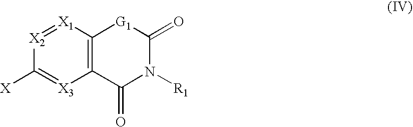

in which X₁, X₂, X₃, G₁, X, and R₁ are as defined hereinbefore,

compound of formula (IV) in which the leaving group X is reacted with a cyanocuprate to yield the compound of formula (V):

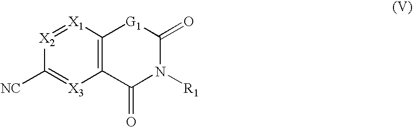

in which X₁, X₂, X₃, G₁, and R₁ are as defined hereinbefore,

which compound of formula (V) is treated with an acid like sulfuric acid to yield the compound of formula (VI):

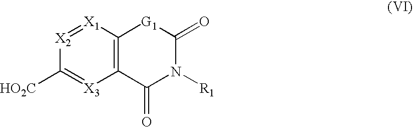

in which X₁, X₂, X₃, G₁, and R₁ are as defined hereinbefore,

compound of formula (VI) which is treated with a compound of formula (VII):

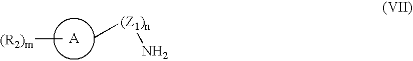

in which Z₁, R₂, A, n and m have the same definitions as the compound of formula (I),

by activating the acid function with an activator, in the presence of diisopropylethylamine and a solvent, to yield the compound of formula (I/a) which is a particular case of the compounds of formula (I):

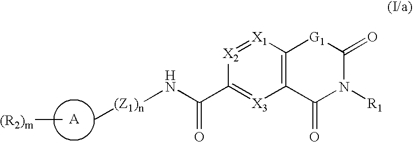

in which X₁, X₂, X₃, G₁, Z₁, R₁, R₂, A, n and m are as defined hereinbefore,

compounds of formula (I/a) constitute, some compounds of the invention, which are purified, where appropriate, according to a conventional purification technique, which are separated, where appropriate, into their different isomers according to a conventional separation technique, and which are converted, where appropriate, into addition salts thereof with a pharmaceutically-acceptable acid or base, or into N-oxide thereof.

The invention also relates to another process for the preparation of specific compounds of formula (I/a), which are a particular case of compounds of formula (I), which uses as starting material a compound of formula (II/A):

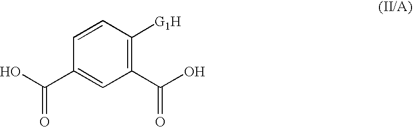

in which G₁ has the same definitions as the compound of formula (I),

compound of formula (II/A) which is treated with SOCl₂ to yield the compound of formula (III/A):

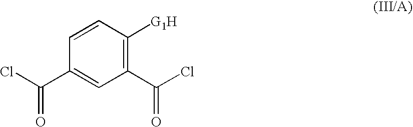

in which G₁ is as defined hereinbefore,

compound of formula (III/A) reacting with a benzylamine derivative of formula (IV/A):

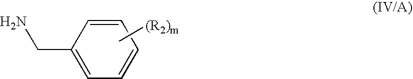

in which R₂ and m are as defined in the compound of formula (I),

to yield the compound of formula (V/A):

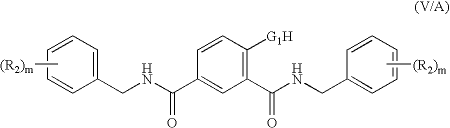

in which G₁, m and R₂ are as defined hereinbefore,

compound of formula (V/A) reacting with a chloroformate compound, to yield the compound of formula (I/c) which is a particular case of the compounds of formula (I):

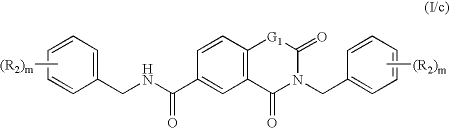

in which G₁, R₂ and m have the same definitions as the compound of formula (I), compounds of formula (I/c) constitute some compounds of the invention, which are purified, where appropriate, according to a conventional purification technique, which are separated, where appropriate, into their different isomers according to a conventional separation technique, and which are converted, where appropriate, into addition salts thereof with a pharmaceutically-acceptable acid or base, or into N-oxide thereof.

The invention also relates to a process for the preparation of compounds of formula (I), which uses as starting material a compound of formula (II):

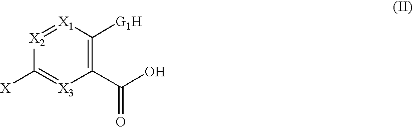

in which X₁, X₂, X₃, and G₁ have the same definitions as the compound of formula (I), and X represents a leaving group selected from halogen, triflate, mesyslate, tosylate and SO₂alkyl,

compound of formula (II) which is treated in basic medium with a benzylisocyanate to yield the compound of formula (VIII):

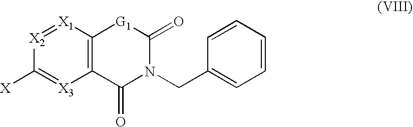

in which X₁, X₂, X₃, G₁, and X are as defined hereinbefore,

compound of formula (VIII) which is treated with AlCl₃ in an apolar solvent to yield the compound of formula (IX):

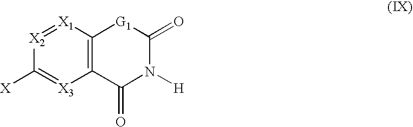

in which X₁, X₂, X₃, G₁, and X are as defined hereinbefore,

which compound of formula (IX) is treated in the presence of an inorganic base with a compound of formula (X):

R₁—X′ (X)

in which R₁ is as defined in the compound of formula (I) and X′ represents a leaving group like halogen atom, mesylate, tosylate or triflate group,

to yield a compound of formula (XI):

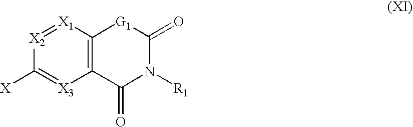

in which X₁, X₂, X₃, G₁, X and R₁ are as defined hereinbefore,

compound of formula (XI) which is condensed, in the presence of dichlorobis(triphenylphosphine)palladium, cupper iodide and N,N′-diisopropylethylamine in dimethylformamide, on a compound of formula (XII):

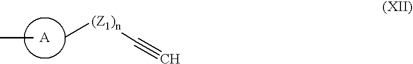

in which Z₁, R₂, A, n and m have the same definitions as the compound of formula (I),

to yield the compound of formula (I/b), which is a particular case of the compound of formula (I):

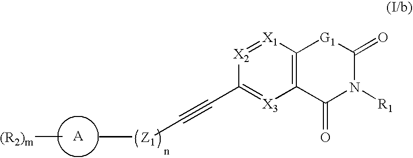

in which X₁, X₂, X₃, G₁, Z₁, R₁, R₂, A, n and m are as defined hereinbefore,

compounds of formula (I/b) constitute some compounds of the invention, which are purified, where appropriate, according to a conventional purification technique, which are separated, where appropriate, into their different isomers according to a conventional separation technique, and which are converted, where appropriate, into addition salts thereof with a pharmaceutically-acceptable acid or base, or into N-oxide thereof.

A general process for the synthesis of the compounds of formula (I) is described in the following scheme:

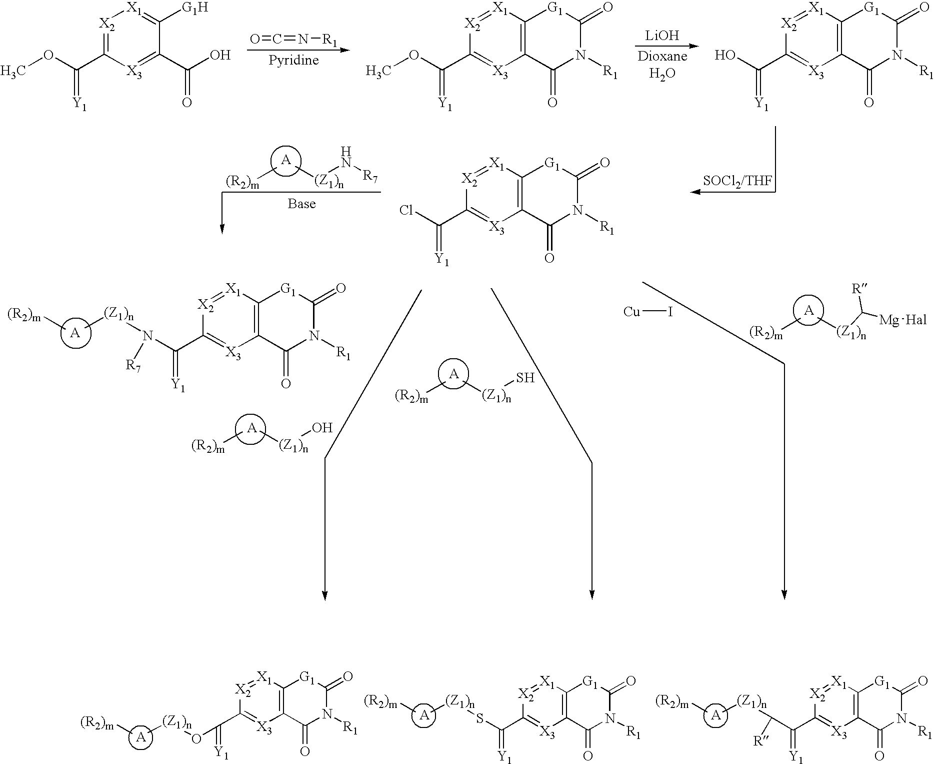

in which R₇ is hydrogen or (C₁-C₆)alkyl, R″ is hydrogen or (C₁-C₆)alkyl, and R₁, R₂, G₁, X₁, X₂, X₃, A, Y₁, Z₁, n and m have the same meaning as that defined for the compound of formula (I).

The compounds of the invention that are present in the form of a mixture of diastereoisomers are isolated in a pure form by using conventional separation techniques such as chromatography.

As mentioned above, compounds of formula (I) of the present invention are matrix metalloprotease inhibitors, and more particularly inhibitors of the enzyme MMP-13.

In this respect, their use is recommended for the treatment of diseases or disorders benefiting from therapy by MMP-13 inhibition. By way of example, the use of the compounds of the present invention may be recommended for the treatment of any pathology in which destruction of extracellular matrix tissue occurs, and most particularly pathologies such as arthritis, rheumatoid arthritis, osteoarthritis, osteoporosis, periodontal diseases, inflammatory bowel disease, psoriasis, multiple sclerosis, cardiac insufficiency, atherosclerosis, asthma, chronic obstructive pulmonary disease, age-related macular degeneration and cancers.

The present invention also relates to pharmaceutical compositions comprising as active ingredient at least one compound of formula (I), an isomer thereof, a N-oxide thereof, or an addition salt thereof with a pharmaceutically-acceptable acid or base, alone or in combination with one or more pharmaceutically-acceptable, inert, non-toxic excipients or carriers.

Among the pharmaceutical compositions according to the invention, there may be mentioned more especially those that are suitable for oral, parenteral (intravenous, intramuscular or subcutaneous), per- or trans-cutaneous, intravaginal, rectal, nasal, perlingual, buccal, ocular or respiratory administration.

Pharmaceutical compositions according to the invention for parenteral injections especially include aqueous and non-aqueous sterile solutions, dispersions, suspension and emulsions, and also sterile powders for reconstituting injectable solutions or dispersions.

Pharmaceutical compositions according to the invention for oral administration in solid form especially include tablets or dragées, sublingual tablets, sachets, gelatin capsules and granules, for oral, nasal, buccal or ocular administration in liquid form, especially include emulsions, solutions, suspensions, drop, syrups and aerosols.

Pharmaceutical compositions for rectal or vaginal administration are preferably suppositories, and those for per- or trans-cutaneous administration especially include powders, aerosols, creams, ointment, gels and patches.

The pharmaceutical compositions mentioned hereinbefore illustrate the invention but do not limit it in any way.

Among the pharmaceutically acceptable, inert, non-toxic excipients or carriers there may be mentioned, by way of non-limiting example, diluents, solvents, preservatives, wetting agents, emulsifiers, dispersing agents, binders, swelling agents, disintegrating agents, retardants, lubricants, absorbents, suspending agents, colorants, aromatizing agents etc . . .

The useful dosage varies according to the age and weight of the patient, the administration route, the pharmaceutical composition used, the nature and severity of the disorder and the administration of any associated treatments. The dosage ranges from 2 mg to 1 g per day in one or more administrations. The compositions are prepared by methods that are common to those skilled in the art and generally comprise 0.5% to 60% by weight of active principle (compound of formula (I)) and 40% to 99.5% by weight of pharmaceutically acceptable excipients or carriers.

The examples that follow illustrate the invention but do not limit it in any way.

The starting materials used are products that are known or that are prepared according to known operating procedures. The various preparations yield synthetic intermediates that are useful in preparation of the compounds of the invention. Some of these intermediates are new compounds.

The structures of the compounds described in the Examples and Preparations were determined according to the usual spectrophotometric techniques (infrared, nuclear magnetic resonance, mass spectrometry, . . . )

In the Examples, it is understood that:

DMSO means dimethylsulfoxide,

TOTU means O-(ethoxycarbonyl)cyanomethylamino]-N—N—N′—N′-tetramethyl uronium fluoroborate,

### EXAMPLES

Intermediate A: 3-benzyl-6-bromo-3,4-dihydro-benzothiazine-2,4-dione

A stirred suspension of 5-bromo-2-mercaptobenzoic acid (prepared after K. Sindelar and coil., Coll. Czech. Chem. Comm., 1988, 53 (2), 340) (8 g, 34.3 mmol) in pyridine (100 ml) was treated with benzyl isocyanate (4.3 ml, 34.3 mmol) and the mixture was heated at 105° C. for 7 hours under a nitrogen atmosphere. Further benzyl isocyanate was added (4.3 ml) and the mixture heated at 105° C. overnight under stirring. After cooling to room temperature, water was added until precipitation and the suspension stirred for 1 hour. The resulting precipitate was collected by filtration, washed several times with water and dried under high vacuum to give 11.5 g (yield: 96%) of the entitled compound as a white amorphous solid.

Intermediate B: 3-benzyl-6-cyano-3,4-dihydro-benzothiazine-2,4-dione

CuCN (0.197 g, 2.2 mmol) was added to a suspension of 3-benzyl-6-bromo-3,4-dihydro-benzothiazine-2,4-dione (intermediate A; 0.35 g, 1.22 mmol) in N-methylpyrrolidone (4 ml) and the suspension obtained was heated at reflux under stirring for 2.5 hours. The solvent was removed under reduced pressure and the sticky residue obtained was stirred in a mixture of NH₄OH solution and dichloromethane. The organic phase was separated, washed with brine and dried over Na₂SO₄. The solvent was evaporated to afford 0.28 g of crude solid that was purified by chromatography on silica gel (cyclohexane 20/CH₂Cl₂ 80) to give the entitled compound (0.15 g; yield: 51%) as a white solid pure in TLC (cyclohexane 20/CH₂Cl₂ 80; Rf=0.40).

Intermediate C: 3-benzyl-6-carboxy-3,4-dihydro-benzothiazine-2,4-dione

A suspension of 3-benzyl-6-cyano-3,4-dihydro-benzothiazine-2,4-dione (intermediate B; 0.12 g, 0.4 mmol) in concentrated sulfuric acid (3 ml) and water (3 ml) was heated at reflux under stirring for 3 hours. After cooling to room temperature, water was added and the insoluble solid was collected by filtration, washed several times with water and dried under high vacuum to give, after purification by chromatography on silica gel (CH₂Cl₂ 95/methanol 5), 0.04 g (yield: 31%) of the entitled compound as a white solid pure in TLC (CH₂Cl₂ 90/methanol 10 ; Rf=0.30).

Intermediate D: 4-hydroxy-N,N′-bis[(4-methoxyphenyl)methyl]-1,3-benzenedicarboxamide

A mixture of 4-hydroxyisophthalic acid (2.0 g; 11 mmol) in thionyl chloride (20 ml) and dimethylformamide (2 drops) was heated at reflux under stirring overnight. The excess of thionyl chloride was removed by evaporation and the residue dissolved into dichloromethane (100 ml).

After cooling, 4-methoxybenzylamine (6.8 g ; 50 mmol) was added in one portion and the mixture obtained was stirred at room temperature for 1 hour. The insoluble solid was separated by filtration and purified by chromatography on silica gel (CH₂Cl₂ 95/methanol 5) to give 2.0 g of the entitled compound (yield: 43%) as a white solid pure in TLC (CH₂Cl₂ 90/methanol 10; Rf=0.70).

Intermediate E: 6-bromo-3,4-dihydro-benzothiazine-2,4-dione

Under an inert atmosphere, aluminium chloride (5.51 g, 41.3 mmol) was added in portions to a suspension of 3-benzyl-6-bromo-3,4-dihydro-benzothiazine-2,4-dione (intermediate A; 2.4 g, 6.89 mmol) in benzene (50 ml) and the mixture obtained was heated at 50° C. under stirring for 2 hours. After cooling, the mixture was poured into iced water, the precipitated product was filtrated after 1 h standing, washed several times with water until neutral pH, dried and finally triturated in dichloromethane then dried under high vacuum to give 1.5 g (yield: 84%) of the entitled compound pure in TLC (CH₂Cl₂; Rf=0.10). NMR H¹ (DMSO) δ (ppm): 5.5(s, 2H); 7.25-7.35 (m, 3H); 7.5 (m, 1H); 7.65 (m, 2H); 8.65 (m, 1H); 8.75 (m, 1H); 9.05 (s, 1H).

Intermediate F: t-butyl 4-(6-bromo-2,4-dioxo-4H-1,3-benzothiazin-3-ylmethyl)-benzoate

A suspension of 6-bromo-3,4-dihydro-benzothiazine-2,4-dione (intermediate E; 1.5 g, 5.8 mmol) and cesium carbonate (1.89 g, 5.8 mmol) in dimethylformamide (20 ml) was stirred under a nitrogen atmosphere for 0.5 hour at room temperature and treated with 4-(t-butoxycarbonyl)benzyl bromide (1.57 g, 5.8 mmol); the mixture obtained was heated at 80° C. under stirring and inert atmosphere for 2 hours. The solvent was removed under reduced pressure, and the residue was partitioned between water and dichloromethane. The aqueous layer was reextracted with CH₂Cl₂, the organic phases combined and dried over Na₂SO₄. The solvent was removed under reduced pressure to afford 2.4 g of crude solid that was purified by chromatography on silica gel (CH₂Cl₂) to give the entitled compound (1.95 g; yield: 85%) as a white solid pure in TLC (CH₂Cl₂ 99/CH₃OH 1; Rf=0.70).

Intermediate G: 4-(6-bromo-2,4-dioxo-4H-1,3-benzothiazin-3-ylmethyl)-benzoic acid

A stirred solution of 6-bromo-3-(4-t-butoxycarbonylbenzyl)-3,4-dihydro-benzothiazine-2,4-dione (intermediate F; 0.6 g, 1.34 mmol) in CH₂Cl₂ (60 ml) was treated at room temperature with trifluoroacetic acid (6 ml). The reaction mixture was stirred overnight at room temperature and poured into water; the resulting insoluble product was isolated by filtration, washed several times until neutral pH and dried under vacuum to afford the entitled acid (0.45 g; yield: 86%) as a white solid pure in TLC (CH₂Cl2-95/CH₃OH 5; Rf=0.35).

### Example 1

3-Benzyl-2,4-dioxo-3,4-dihydro-2H-benz[e][1,3]thiazine-6-carboxylic acid 4-methoxy benzylamide

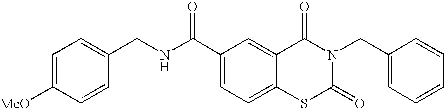

To a solution of 25 mg (0.08 mmol) of intermediate C in 2 ml of dimethylformamide, 10.9 mg (0.08 mmol) of 4-methoxybenzylamine and 26 mg (0.08 mmol) of TOTU were added under stirring. After external cooling with ice bath, 20 mg (0.16 mmol) of N,N-diisopropyl-N-ethylamine were added and the yellow resulting solution was stirred overnight at room temperature. The solvent was removed under vacuum and the residual brown oil was purified by column chromatography over silica gel (dichloromethane then dichloromethane/methanol : 99.5/0.5) to yield 13 mg of the desired product (yield : 38%).

N.M.R (CDCl₃) ¹H δ (ppm): 3.8 (s, 3H); 4.6 (d, 2H); 5.35 (s, 2H); 6.5 (s, 1H); 6.9 (d, 2H); 7.2-7.35 (m, 5H); 7.4 (d, 2H) ; 7.5 (d, 2H); 8.15 (d, 1H); 8.6 (s, 1H).

IR: 1649, 1543, 1514, 1406, 1284, 1253, 1231, 1185, 1145, 1030, 824, 731 cm⁻¹

HPLC: Purity=96%

### Example 2

3-Benzyl-2,4dioxo-3,4-dihydro-2H-benzo[e][1,3]oxazine-6-carboxylic acid 4-methoxy benzylamide

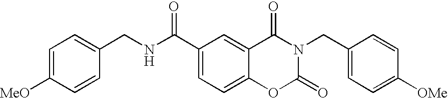

A cooled solution of N, N′-bis-(4-methoxybenzyl)-4-hydroxyisophthalic acid (intermediate D, 0.42 g, 1 mmol) in pyridine (5 ml) and acetonitrile (3 ml) was treated with ethyl chloroformate (0.12 g, 1.1 mmol) under stirring and the mixture was heated at 120° C. for 8 hours under a nitrogen atmosphere. Further ethyl chloroformate was added (1.1 ml) and the mixture heated at 120° C. overnight under stirring. After cooling to room temperature, the reaction mixture was poured into diluted hydrochloric solution and the product extracted several times with dichloromethane. The joined organic phases were washed with diluted hydrochloric solution, diluted solution of sodium hydroxide and brine successively and dried over Na₂SO₄. The solvent was evaporated and the residue triturated in dichloromethane; the insoluble solid is filtrated and dried to afford the entitled compound (0.32 g ; yield: 71%) as a white solid pure in TLC (CH₂Cl₂ 95/methanol ; Rf=0.40).

N.M.R (DMSO-d₆) ¹H δ (ppm): 3.7 (s, 6H); 4.4 (d, 2H); 6.8-6.9 (m, 4H); 7.25 (d, 2H); 7.3 (d, 2H); 7.5 (d, 1H); 8.25 (d, 1H); 8.5 (s, 1H); 9.25 (t, 1H).

IR: 1759, 1693, 1638, 1513, 1446, 1327, 1305, 1244 cm⁻¹

MP=157° C.

HPLC: Purity 98.5%

### Example 3

4-[2,4-Dioxo-6-(3-phenyl-prop-1ynyl)-4H-1,3-benzothiazin-3-ylmethyl]-benzoic acid

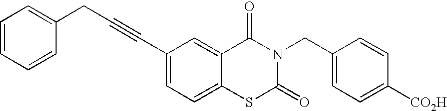

6-bromo-3-(4-carboxybenzyl)-3,4-dihydro-benzothiazine-2,4-dione (Intermediate G) (0.39 g; 0.994 mmol) in dimethylformamide (4 ml) was stirred at room temperature under nitrogen atmosphere and N-ethyl-N,N-diisopropylamine (0.51 g, 3.97 mmol) was added; the mixture was stirred until complete solubilisation. At this time, 3-phenylprop-1-yne (0.16 g; 1.39 mmol) was added followed by PdCl₂(PPh₃)₂ (30 mg) and a catalytic amount of CuI. The mixture obtained was heated to 50° C. under nitrogen atmosphere and maintained under stirring for 3 hours. After cooling, the solvent was removed under reduced pressure and the semi-solid residue obtained was stirred in a mixture of water and dichloromethane for 25 minutes. The solid insoluble in the 2 phases was isolated by filtration, washed with CH₂Cl₂ and dried under vacuum to afford a first portion (0.16 g) of the entitled compound. The organic phase was separated, washed with brine, dried over Na₂SO₄ and evaporated to give an additional portion (0.23 g) of the desired compound (yield 92%).

N.M.R (DMSO-d₆) ¹H δ (ppm): 3.94 (s, 2H); 5.23 (s, 2H); 7.27 (t, 1H); 7.37 (t, 2H); 7.40-7.50 (m, 4H); 7.67 (d, 1H); 7.84-7.95 (m, 2H); 8.23 (s, 1H); 12.75-13.05 (m, 1H).

IR: 1690, 1638, 1425, 1408, 1341, 1318, 1297, 1286, 1181, 1149, 913, 768, 726, 707 cm⁻¹

MP=240-242° C.

HPLC: Purity=98%

Pharmacological Studies of Compounds of the Invention

### Example 4

Evaluation of the In Vitro Activity of the MMP-13 Inhibitor Compounds According to the Invention.

The inhibitory activity of the compounds of formula (I) according to the invention with respect to matrix metalloprotease-13 is evaluated by testing the ability of the compounds of the invention to inhibit the proteolysis of a peptide substrate with MMP-13.

The peptide substrate used in the test is the following peptide: Ac-Pro-Leu-Gly-thioester-Leu-Leu-Gly-OEt.

The inhibitory activity of a compound of formula (I) according to the invention is expressed as the IC₅₀ value, which is the concentration of inhibitor for which an inhibition of 50% of the activity of the matrix metalloprotease under consideration is observed.

To carry out this test, a reaction medium of 100 μt volume is prepared, containing: 50 mM of HEPES buffer, 10 mM of CaCl₂ and 1 mM of 5,5′-dithiobis-(2-nitrobenzoic acid) (DTNB), and 100 μM of substrate, the pH being adjusted to 7.0.

Increasing concentrations of the inhibitory compound present in a 2.0% DMSO solution and 2.5 nM of the catalytic domain of human MMP-13 are added to the test samples.

The concentrations of inhibitors present in the test samples range from 100 μM to 0.5 nM. The measurement of the proteolysis of the substrate peptide is monitored by measuring the absorbance at 405 nm using a spectrophotometer for reading microplates, at the laboratory temperature, the measurements being carried out continuously for 10 to 15 minutes.

The IC₅₀ values are calculated from a curve in which the percentage of the catalytic activity relative to the control is represented on the X-axis and the concentration of inhibitor is represented on the Y-axis.

The IC₅₀ values on MMP-13 of the compounds of Examples 1, 2 and 3 are respectively 0.037 μM, 0.063 μM, and 0.0012 μM.

The test described above for the inhibition of MMP-13 was also adapted and used to determine the ability of, the compounds of formula (I) to inhibit the matrix metalloproteases MMP-1, MMP-2, MMP-3, MMP-7, MMP-9, MMP-12 and MMP-14.

The results obtained show that the compounds according to the invention generally have IC₅₀ values for MMP-13 which are about 100 times lower than the IC₅₀ values for the same compounds with respect to the other matrix metalloproteases tested. Then the IC₅₀ values on MMP-1, MMP-2, MMP-3, MMP-7, MMP-9, MMP-12 and MMP-14 for the compound of Example 1 are respectively 30 μM, 100 μM, 18 μM, 30 μM, 100 μM, 100 μM, and 100 μM.

What is claimed is:

1- A compound of formula (I): 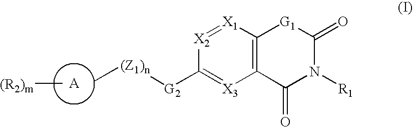
wherein:
X₁, X₂, and X₃, independently of each other, represent a nitrogen atom or a group —CR₃ in which R₃ represents a group selected from hydrogen, (C₁-C₆)alkyl, amino, mono(C₁-C₆)alkylamino, di(C₁-C₆)alkylamino, hydroxy, (C₁-C₆)alkoxy, and halogen, it being understood that not more than two of the groups X₁, X₂ and X₃ simultaneously represent a nitrogen atom,
G₁ represents an oxygen atom or a group S(O)ₚ in which p represents an integer from 0 to 2 inclusive,
G₂ represents a group selected from carbon-carbon triple bond, C═O, C═S, S(O)_{q} in which q represents an integer from 0 to 2 inclusive, or a group of formula (i/a): 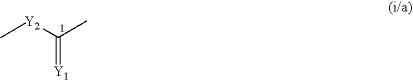
in which the carbon atom with the number 1 is attached to the bicycle of the compound of formula (I), Y₁ represents a group selected from oxygen, sulphur, —NH and —N(C₁-C₆)alkyl, and Y₂ represents a group selected from oxygen, sulphur, —NH and —N(C₁-C₆)alkyl,
n represents an integer from 0 to 6 inclusive,
Z₁ represents —CR₄R₅, wherein R₄ and R₅, identical or different independently of each other, represent a group selected from hydrogen, (C₁-C₆)alkyl, trihalogeno(C₁-C₆)alkyl, halogen, amino, mono(C₁-C₆)alkylamino, di(C₁-C₆)alkylamino in which each alkyl moiety is identical or different, —OR₆, —SR₆, and —C(═O)OR₆, in which R₆ is hydrogen atom or (C₁-C₆)alkyl, and
wherein when n is greater than or equal to 2, the hydrocarbon chain Z₁ optionally contains one to two isolated or conjugated multiple bonds,
and/or wherein when n is greater than or equal to 2 one of said —CR₄R₅ may be replaced with a group selected from oxygen, S(O)ᵣ in which r represents an integer from 0 to 2 inclusive, —NH and —N(C₁-C₆)alkyl,
A represents a group selected from aryl, heteroaryl, cycloalkyl, and heterocycloalkyl, these groups being a 5- or 6-membered monocycle, or bicycle itself composed of two 5- or 6-membered monocycles,
R₁ represents a group selected from:
hydrogen,
(C₁-C₆)alkyl, (C₂-C₆)alkenyl, (C₂-C₆)alkynyl, these groups may be optionally substituted with one or more groups, which may be identical or different independently of each other, selected from amino, cyano, trihalogeno(C₁-C₆)alkyl, cycloalkyl, —C(═O)NR₇R₈, —C(═O)OR₇, OR₇, and SR₇, in which R₇ and R₈, which may be identical or different independently of each other, represent hydrogen or (C₁-C₆)alkyl,
and the group of formula (i/b): 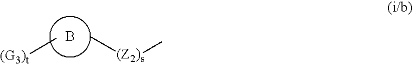
in which s is an integer from 0 to 8 inclusive,
Z₂ represents —CR₉R₁₀ wherein R₉ and R₁₀, identical or different independently of each other, represent a group selected from hydrogen, (C₁-C₆)alkyl, phenyl, trihalogeno(C₁-C₆)alkyl, halogen, amino, OR₆, SR₆ and —C(═O)OR₆ in which R₆ is as defined hereinbefore, and
wherein when s is greater than or equal to 2, the hydrocarbon chain Z₂ optionally contains one or two isolated or conjugated multiple bonds,
and/or wherein when p is greater or equal to 2, one of said —CR₉R₁₀ may be replaced with a group selected from oxygen, S(O)ᵤ in which u is an integer from 0 to 2 inclusive, —NH, —N(C₁-C₆)alkyl, and carbonyl,
B represents a group selected from aryl, heteroaryl, cycloalkyl, and heterocycloalkyl, these groups being a 5- or 6-membered monocycle, or bicycle itself composed of two 5- or 6-membered monocycles,
t is an integer from 0 to 7 inclusive,
the group(s) G₃, which may be identical or different independently of each other, is (are) selected from (C₁-C₆)alkyl, halogen, CN, NO₂, CF₃, OCF₃, —(CH₂)ₖNR₁₁R_{12, —N(R}₁₁)C(═O)R₁₂, —N(R₁₁)C(═O)OR₁₂, —N(R₁₁)SO₂R₁₂, —N(SO₂R₁₁)₂, —OR₁₁, —S(O)ₖ₁R₁₁, —SO₂—N(R₁₁)—(CH₂)ₖ₂—NR₁₂R₁₃, —(CH₂)ₖSO₂NR₁₁R₁₂, —X₄(CH₂)ₖC(═O)OR₁₁, —(CH₂)ₖC(═O)OR₁₁, —C(═O)O—(CH₂)ₖ₂—NR₁₁R₁₂, —C(═O)O—(CH₂)ₖ₂—C(═O)OR₁₄, —X₄(CH₂)ₖC(═O)NR₁₁R₁₂, —(CH₂)ₖC(═O)NR₁₁R₁₂, —R₁₅—C(═O)OR₁₁, —X₅—R₁₆, and —C(═O)—R₁₇—NR₁₁R₁₂ in which:
X₄ represents a group selected from oxygen, sulphur optionally substituted by one or two oxygen, and nitrogen substituted by a hydrogen or a (C₁-C₆)alkyl group,
k is an integer from 0 to 3 inclusive,
k1 is an integer from 0 to 2 inclusive,
k2 is an integer from 1 to 4 inclusive,
R₁₁, R₁₂ and R₁₃, which may be identical or different independently of each other, are selected from hydrogen and (C₁-C₆)alkyl,
R₁₄ represents a group selected from (C₁-C₆)alkyl, —R₁₇—NR₁₁R₁₂, —R₁₇—NR₁₁—C(═O)—R₁₇—NR₁₂R₁₃, and —C(═O)O—R₁₇—NR₁₁R₁₂ in which R₁₇ represents a linear or branched (C₁-C₆)alkylene group, and R₁₁, R₁₂ and R₁₃ are as defined hereinbefore,
R₁₅ represents a (C₃-C₆)cycloalkyl group,
X₅ represents a group selected from a single bond, —CH₂—, oxygen, sulphur optionally substituted by one or two oxygen, and nitrogen substituted by hydrogen or (C₁-C₆)alkyl,
R₁₆ represents a group selected from:
a 5- or 6-membered monocyclic aryl or heteroaryl, which is optionally substituted by one or more groups, which may be identical or different independently of each other, selected from (C₁-C₆)alkyl, halogen, hydroxy, cyano, tetrazolyl, amino, and —C(═O)OR₇ wherein R₇ represents hydrogen or (C₁-C₆)alkyl,
and a 5- or 6-membered monocyclic cycloalkyl or heterocycloalkyl, which is optionally substituted by one or more groups, which may be identical or different independently of each other, selected from (C₁-C₆)alkyl, halogen, hydroxy, oxo, cyano, tetrazolyl, amino, and -C(═O)OR₇ wherein R₇ represents hydrogen or (C₁-C₆)alkyl,
m is an integer from 0 to 7 inclusive,
the group(s) R₂, which may be identical or different independently of each other, is (are) selected from (C₁-C₆)alkyl, halogen, —CN, —NO₂, —SCF₃, —CF₃, —OCF₃, —NR₇R₈, —OR₇, —SR₇, —SOR₇, —SO₂R₇, —(CH₂)ₖSO₂NR₇R₈, —X₇(CH₂)ₖC(═O)OR₇, —(CH₂)ₖC(═O)OR₇, —X₇(CH₂)ₖC(═O)NR₇R₈, —(CH₂)ₖC(═O)NR₇R₈, and —X₈—R₁₈ in which:
X₇ represents a group selected from oxygen, sulphur optionally substituted by one or two oxygen, and nitrogen substituted by hydrogen or (C₁-C₆)alkyl,
k is an integer from 0to 3 inclusive,
R₇ and R₈, which may be identical or different independently of each other, are selected from hydrogen and (C₁-C₆)alkyl,
X₈ represents a group selected from single bond, —CH₂—, oxygen, sulphur optionally substituted by one or two oxygen, and nitrogen substituted by hydrogen or (C₁-C₆)alkyl,
R₁₈ represents a group selected from phenyl, a 5- or 6-membered monocyclic, heteroaryl, and a 5- or, 6-membered monocyclic cycloalkyl, each of these groups being optionally substituted by one or more groups, which may be identical or different independently of each other, selected from (C₁-C₆)alkyl, halogen, hydroxy and amino,
or a racemic form, isomer, N-oxide, or pharmaceutically acceptable salt thereof, wherein the compound of formula (I) is not 6-(2,4-dioxo-3,4-dihydro-2H-1,3-benzothiazine)-benzoate, 6-phenylthio-2,4-dioxo-3,4-dihydro-2H-1,3-benzothiazine, 6-benzylsulphonyl-2,4-dioxo-3,4-dihydro-2H-1.3-benzothiazine, 6-benzophenone-2,4-dioxo-3,4-dihydro-2H-1,3-benzothiazine or 6-(2,4-dihydroxy)-benzophenone-2,4-dioxo-3,4-dihydro-2H-1,3-benzothiazine.

2- The compound according to claim 1, or a racemic form, isomer, N-oxide, or pharmaceutically acceptable salt thereof, wherein
G₁ represents a sulphur atom,
G₂ represents a group of formula (i/a): 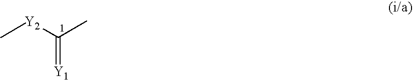
in which the carbon atom with the number 1 is attached to the bicycle of the compound of formula (I), Y₁ represents an oxygen atom, and Y₂ represents a group —NH, X₁, X₂, X₃, n, Z₁, A, R₁, m and R₂ are as defined in formula (I).

3- The compound according to claim 1, or a racemic form, isomer, N-oxide, or pharmaceutically acceptable salt thereof, wherein
G₁ represents an oxygen atom,
G₂ represents a group of formula (i/a): 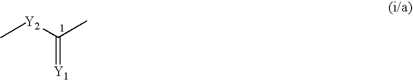
in which the carbon atom with the number 1 is attached to the bicycle of the compound of formula (I), Y₁ represents an oxygen atom, and Y₂ represents a group —NH, X₁, X₂, X₃, n, Z₁, A, R₁, m and R₂ are as defined in formula (I).

4- The compound according to claim 1, or a racemic form, isomer, N-oxide, or pharmaceutically acceptable salt thereof, wherein
G₁ represents a sulphur atom,
G₂ represents a carbon-carbon triple bond,
n represents an integer from 1 to 6 inclusive,
X₁, X₂, X₃, Z₁, A, R₁, m and R₂ are as defined in formula (I).

5- The compound according to claim 1, or a racemic form, isomer, N-oxide, or pharmaceutically acceptable salt thereof, wherein
G₁ represents an oxygen atom,
G₂ represents a carbon-carbon triple bond,
n represents an integer from 1 to 6 inclusive,
X₁, X₂, X₃, Z₁, A, R₁, m and R₂ are as defined in formula (I).

6- The compound according to claim 1, or a racemic form, isomer, N-oxide, or pharmaceutically acceptable salt thereof, wherein R₁ represents a group of formula (i/b): 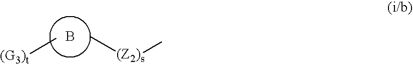
wherein Z₂, s, B, G₃ and t are as defined in the compound of formula (I).

7- The compound according to claim 6, or a racemic form, isomer, N-oxide, or pharmaceutically acceptable salt thereof, wherein R₁ represents a group of formula (i/b): 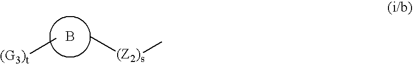
wherein Z₂ represents a group —CR₉R₁₀ in which R₉ and R₁₀ represents each a hydrogen atom, s is equal to one, and B, G₃, and t are as defined in the compound of formula (I).

8- The compound according to claim 7, or a racemic form, isomer, N-oxide, or pharmaceutically acceptable salt thereof, wherein R₁ represents a group of formula (i/b): 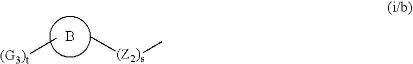
wherein B represents a phenyl group, t is equal to 0 or 1, and G₃, when it is present, represents a group selected from OR₁₁, halogen, and (CH₂)ₖC(═O)OR₁₁ in which R₁₁ represents an hydrogen atom or a (C₁-C₆)alkyl group and k is equal to zero.

9- The compound according to claim 1, or a racemic form, isomer, N-oxide, or pharmaceutically acceptable salt thereof, wherein X₁, X₂, and X₃ represent each a group —CR₃ in which R₃ represents a hydrogen atom.

10- The compound according to claim 1, or a racemic form, isomer, N-oxide, or pharmaceutically acceptable salt thereof, wherein X₁ represents a group —CR₃ in which R₃ represents a hydrogen atom, X₂ represents a nitrogen atom or a group —CR₃ in which R₃ represents a hydrogen atom, and X₃ represents a group —CR₃ in which R₃ represents a hydrogen atom.

11- The compound according to claim 1, or a racemic form, isomer, N-oxide, or pharmaceutically acceptable salt thereof, wherein Z₁ represents —CR₄R₅ in which R₄ and R₅ represent each a hydrogen atom, and n is equal to one.

12- The compound according to claim 1, or a racemic form, isomer, N-oxide, or pharmaceutically acceptable salt thereof, wherein A represents a phenyl group, m is equal to zero or one, and R₂ represents a (C₁-C₆)alkoxy group or a hydrogen atom.

13- The compound according to claim 1, or a racemic form, isomer, N-oxide, or pharmaceutically acceptable salt thereof, wherein A represents a pyridyl group, m is equal to zero or one, and R₂ represents a (C₁-C₆)alkoxy group or a hydrogen atom.

14- The compound according to claim 1, or a racemic form, isomer, N-oxide, or pharmaceutically acceptable salt thereof, wherein A represents an imidazolyl group.

15- The compound according to claim 1 selected from:
3-benzyl-2,4-dioxo-3,4-dihydro-2H-benzo[e][1,3]thiazine-6-carboxylic acid 4-methoxy benzylamide;
3-(4-methoxybenzyl)2,4-dioxo-3,4-dihydro-2H-benzo[e][1,3]oxazine-6-carboxylic acid 4-methoxybenzylamide;
and 4-[2,4-dioxo-6-(3-phenyl-prop-1-ynyl)-4H-1,3-benzothiazin-3-ylmethyl]-benzoic acid; or
a racemic form, isomer, N-oxide, or pharmaceutically acceptable salt thereof.

16- A method for treating a patient afflicted with a disease or disorder that is mediated by a MMP-13 enzyme, comprising administering to the patient an effective amount of a compound of claim 1, or a racemic form, isomer, N-oxide, or pharmaceutically acceptable salt thereof.

17- The method according to claim 16, wherein the disease or disorder is selected from arthritis, rheumatoid arthritis, osteoarthritis, osteoporosis, periodontal diseases, inflammatory bowel disease, psoriasis, multiple sclerosis, cardiac insufficiency, atherosclerosis, asthma, chronic obstructive pulmonary disease, age-related macular degeneration, and cancer.

18- The method according to claim 17, wherein the disease or disorder is arthritis.

19- The method according to claim 18, wherein the disease or disorder is rheumatoid arthritis or osteoarthritis.

20- A pharmaceutical composition comprising as active ingredient an effective amount of a compound as claimed in claim 1, in combination with a pharmaceutically acceptable excipient or carrier.

21- The pharmaceutical composition according to claim 20, wherein the compound as claimed in claim 1 is a compound according to claim 2 or 4.
